(19) United States
(12) Patent Application Publication
(10) Pub. No.: US 2009/0233987 A1
(43) Pub. Date: Sep. 17, 2009

(54) Method and apparatus for drug delivery to tissue or organ for transplant

(75) Inventors: Minoru Obara, Yokohama-Shi (JP); Mitsuhiro Terakawa, Yokohama-Shi (JP); Shunichi Sato, Saitama (JP); Daizoh Saitoh, Tokorozawa-Shi (JP)

Correspondence Address: BIRCH STEWART KOLASCH & BIRCH, PO BOX 747, FALLS CHURCH, VA, 22040-0747, US

(73) Assignee: Keio University, Tokyo (JP)

(21) Appl. No.: 11/878,934

(22) Filed: Jul. 27, 2007

(30) Foreign Application Priority Data
Jul. 28, 2006 (JP) 2006-206101

(51) Int. Cl. A61K 31/7088; A01N 1/02; A61N 5/067

(52) U.S. Cl. 514/44 R; 435/1.1; 435/284.1; 604/20

(57) ABSTRACT

It is intended to provide a method for delivering a drug such as a gene to a tissue or organ for transplant such as skin by use of a laser-induced stress wave (LISW) and thereby producing a high-performance tissue or organ for transplant with a high survival ability and to provide an apparatus for carrying out the method as well as a high-performance tissue or organ for transplant produced by the method. The present invention provides a method for delivering a drug to a graft of a tissue or organ for transplant, comprising applying a drug to a graft of a tissue or organ for transplant and applying, to the graft, a stress wave induced by the laser light irradiation of a light absorber provided in proximity to the graft, wherein the light absorber is made of a substance capable of absorbing a laser light and generating a stress wave.

### BACKGROUND OF THE INVENTION

1. Field of the Invention

The present invention relates to a method for delivering a drug such as a gene to a tissue or organ for transplant such as skin and to an apparatus for delivering a drug such as a gene to a tissue or organ for transplant.

2. Background Art

Foreign molecule delivery into cells, as typified by gene delivery, has been an important technique in medicine and biology in recent years. While a large number of approaches for foreign molecule delivery to cells have been reported, a chemical or physical foreign molecule delivery method without use of viruses has received attention because of its excellent safety to organisms, compared with a virus method. In particular, a method using a laser has been expected to be applied to endoscopes or catheters by use of a high space control property resulting from changes in beam diameter, or optical fibers.

As specific examples, (a) a method using a tiny perforation, (b) a method using the interaction between a laser light and a dye, and (c) a method using pulse laser irradiation into a solution, shown below, have been developed as drug delivery methods using a laser.

### (a) Method Using a Tiny Perforation

This method involves concentrating a laser light onto a cell membrane, transiently making a tiny hole, and thereby delivering a foreign molecule by means of a concentration gradient (see U. K. Tirlapur and K. Konig, Nature, July 2002, Vol. 418, pp. 290-291).

### (b) Method Using the Interaction Between a Laser Light and a Dye

This method involves providing a cell in a dye-containing solution, locally increasing a temperature by the laser light irradiation of the dye near the cell membrane, and thereby denaturing the cell membrane (see G. Palumbo et al., Journal of Photochemistry and Photobiology B, October 1996, Vol. 36, No. 1, pp. 41-46).

(c) Method Using Pulse Laser Irradiation into a Solution

This method involves providing a cell in a solution, gel, or gel surface containing a substance to be delivered, irradiating the solution or gel or the cell with a pulse laser concentrated thereonto, and altering the cell membrane by a shock wave caused thereby (see JP Patent Publication (Kokai) No. 2005-168495A).

In addition, (d) a method using a stress wave (pressure wave) induced by the pulse laser irradiation of a solid material has been developed.

### (d) Method Using a Stress Wave (Pressure Wave) Induced by the Pulse Laser Irradiation of a Solid Material

This method involves generating a stress wave by the pulse laser irradiation of an absorbing solid substance and thereby delivering a foreign molecule to many cells by single treatment (see S. Lee and A. G. Doukas, IEEE Journal of Selected Topics in Quantum Electronics, 1999, Vol. 5, No. 4, pp. 997-1003 and M. Terakawa et al., Optics Letters, June 2004, Vol. 29, No. 11, pp. 1227-1229 and U.S. Pat. Nos. 5,658,892, 6,689,094 and 6,562,004).

In tissue or organ transplantation, a tissue or organ with reduced or lost function is removed and replaced by a tissue or organ that plays a normal or auxiliary role. In transplantation, a tissue or organ provided by a donor is often used, and the development of a tissue or organ created in a bioengineering manner has progressed rapidly in recent years. For example, artificial epidermis, artificial dermis, and artificial skin have been developed for skin transplantation. Alternatively, a skin for transplant enhanced in growth factor generation by delivering, by use of a virus, a gene encoding a particular growth factor functioning in the skin for transplant has also been reported (see Gulsun Erdag et al., Molecular Therapy, Vol. 10, No. 1, pp. 76-85, 2004).

Moreover, a method for in vivo pinpoint gene delivery to a rat by use of a pulse laser-induced stress wave has been reported (see Terakawa et al., Proc. Of SPIE Vol. 6078 60780T-1 and Terakawa et al., Optical Society of Japan, proceedings of Fourth Symposium on Biomedical Optics, p. 26-27, 2005).

An object of the present invention is to provide a method for delivering a drug such as a gene to a tissue or organ for transplant such as skin by use of a laser-induced stress wave (LISW) and thereby producing a high-performance tissue or organ for transplant with a high survival ability and to provide an apparatus for carrying out the method as well as a high-performance tissue or organ for transplant produced by the method.

### SUMMARY OF THE INVENTION

A transplanted tissue or organ generally has a less fast engraftment speed and a low survival rate, though they differ depending on the type of the tissue or organ. Thus, tissue or organ transplantation still has problems. An incompletely engrafted tissue or organ poses a high risk for infectious disease. Therefore, a tissue or organ for transplant with a fast engraftment speed and a high survival rate has been demanded. Graft survival is largely influenced by the angiogenesis of the graft. However, for example, for the skin, blood vessel suture is not carried out during transplantation. Therefore, graft survival in the skin had to depend on a spontaneous cure process. On the other hand, a tissue and organ for transplant that have received gene delivery using a virus vector were characterized by having a high cell growth ability but presented a safety problem in use.

Although some reports have described a method for in vivo gene delivery to a rat by use of a laser-induced stress wave, no report has described a method for gene delivery to a graft of a tissue or organ for transplant.

The present inventor has conducted diligent studies on a method for delivering a gene into a tissue or organ for transplant by use of a stress wave (pressure wave) induced by the laser light irradiation of a light absorber and has consequently completed the present invention by finding out that a drug such as a gene can be delivered efficiently to a graft of a tissue or organ for transplant by applying the drug such as a gene to the graft of a tissue or organ for transplant by use of a pulse laser light with a wavelength of 180 nm to 20 μm, a laser fluence of 0.1 mJ/cm² to 10 J/cm², and a pulse width of 500 fs to 500 ns and applying, to the graft, a stress wave generated by the irradiation of a target comprising a light absorber or the light absorber combined with a transparent material with the laser light.

The present inventions are as follows.

[1] A method of producing a graft of a tissue or organ for transplant having promoted survival ability which comprises delivering a drug to an animal tissue or organ in vitro by applying the drug to the tissue or organ, irradiating a pulse laser light to a light absorber which is provided in proximity to the tissue or organ to induce a stress wave and applying the stress wave to the tissue or organ, wherein the drug is selected from the group consisting of a gene encoding a protein having an angiogenic effect, a gene encoding a protein having an anti-infective effect, a gene encoding a protein having a nerve regenerative effect, a gene encoding a protein having a cell growth effect, and a gene encoding a protein having a hematopoietic effect.
[2] The method of producing a graft of a tissue or organ for transplant having promoted survival ability according to [1] above, wherein the drug is chemically modified with cationic polymer or cationic liposome.
[3] The method of producing a graft of a tissue or organ for transplant having promoted survival ability according to [1] above, wherein the animal tissue or organ is selected from the group consisting of skin, liver, kidney, lung, small intestine, pancreas, and cornea collected from the animal body.
[4] The method of producing a graft of a tissue or organ for transplant having promoted survival ability according to [1] above, wherein the light absorber is selected from the group consisting of rubber, resin or metal.
[5] The method of producing a graft of a tissue or organ for transplant having promoted survival ability according to [1] above, wherein the light absorber is used in combination with a transparent material.
[6] The method of producing a graft of a tissue or organ for transplant having promoted survival ability according to [5] above, wherein the transparent material is selected from the group consisting of a transparent resin, glass, gel and liquid.
[7] The method of producing a graft of a tissue or organ for transplant having promoted survival ability according to [1] above, wherein the graft is treated for making incisions or holes such that the drug can be easily penetrated into the graft.
[8] A graft of a tissue or organ for transplant produced by delivering a drug to the tissue or organ by the method of producing a graft of a tissue or organ for transplant having enhanced survival ability according to any one of [1] to [7] above, wherein the drug is selected from the group consisting of a gene encoding a protein having an angiogenic effect, a gene encoding a protein having an anti-infective effect, a gene encoding a protein having a nerve regenerative effect, a gene encoding a protein having a cell growth effect, and a gene encoding a protein having a hematopoietic effect and the gene is incorporated in an expressible manner to the tissue or organ.
[9] The graft of a tissue or organ for transplant according to [8] above, wherein the angiogenic ability is promoted.
[10] The graft of a tissue or organ for transplant according to [8] above, wherein the survival ability is promoted.
[11] The graft of a tissue or organ for transplant according to [8] above, wherein the tissue or organ is selected from the group consisting of skin, liver, kidney, lung, small intestine, pancreas, and cornea collected from the animal body.
[12] An apparatus for delivering a drug to a graft of a tissue or organ comprising a drug to be delivered to the graft of the tissue or organ,
a target comprising a light absorber or the light absorber combined with a transparent material, a means for irradiating a laser light,
wherein laser light is irradiated to the target to generate a stress wave from the light absorber, and applying the stress wave to the graft of the tissue or organ to deliver the drug to the graft of the tissue or organ and the drug is selected from the group consisting of a gene encoding a protein having an angiogenic effect, a gene encoding a protein having an anti-infective effect, a gene encoding a protein having a nerve regenerative effect, a gene encoding a protein having a cell growth effect, and a gene encoding a protein having a hematopoietic effect.
[13] The apparatus for delivering a drug to a graft of a tissue or organ according to [12] above, further comprising an optical fiber having a terminal case at the terminus of the optical fiber, wherein the terminal case has the target on the bottom of the case in which the transparent material is set on the light absorber.
[14] The apparatus for delivering a drug to a graft of a tissue or organ according to [12] or [13] above, wherein the drug is chemically modified with cationic polymer or cationic liposome.
[15] The apparatus for delivering a drug to a graft of a tissue or organ according to [12] or [13] above, wherein the light absorber is selected from the group consisting of rubber, resin or metal.
[16] The apparatus for delivering a drug to a graft of a tissue or organ according to [12] or [13] above, wherein the light absorber is used in combination with a transparent material.
[17] The apparatus for delivering a drug to a graft of a tissue or organ according to [12] or [13] above, wherein the transparent material is selected from the group consisting of a transparent resin, glass, gel and liquid.
[18] The apparatus for delivering a drug to a graft of a tissue or organ according to [12] or [13] above, wherein the animal tissue or organ is selected from the group consisting of skin, liver, kidney, lung, small intestine, pancreas, and cornea collected from the animal body.
[19] An apparatus for delivering a drug to a tissue or organ in vivo comprising a target comprising a light absorber or the light absorber combined with a transparent material, a means for irradiating a laser light,
an optical fiber having a terminal case at the terminus of the optical fiber,
wherein the terminal case has the target on the bottom of the case and the transparent material is set on the light absorber and the drug is selected from the group consisting of a gene encoding a protein having an angiogenic effect, a gene encoding a protein having an anti-infective effect, a gene encoding a protein having a nerve regenerative effect, a gene encoding a protein having a cell growth effect, and a gene encoding a protein having a hematopoietic effect.
[20] A method for delivering a drug to a tissue or organ in vivo using the apparatus of [19] above, which comprises applying a drug to the tissue or organ and irradiating laser light to the tissue or organ in vivo, wherein the drug is selected from the group consisting of a gene encoding a protein having an angiogenic effect, a gene encoding a protein having an anti-infective effect, a gene encoding a protein having a nerve regenerative effect, a gene encoding a protein having a cell growth effect, and a gene encoding a protein having a hematopoietic effect.

The method for drug delivery to a tissue or organ for transplant according to the present invention can deliver a drug such as a gene to the whole tissue or organ for transplant including a deep region of the tissue or organ for transplant. A gene encoding an active substance having an angiogenic effect or anti-infective effect can be delivered as a drug to thereby obtain a graft of a high-performance tissue or organ for transplant having angiogenic function or anti-infective function.

As shown in Example, the delivery of a gene having an angiogenic effect can give a tissue or organ for transplant enhanced in angiogenesis, leading to promoted graft survival. For example, for skin grafting, transplantation at an early stage can produce effects such as the prevention of infection, the prevention of fluid loss, and the relief of pain.

### BRIEF DESCRIPTION OF THE DRAWINGS

FIG. 1 is a diagram showing a method for delivering a drug such as a gene to a tissue or organ for transplant (No. 1);

FIG. 2 is a diagram showing a method for delivering a drug such as a gene to a tissue or organ for transplant (No. 2);

FIG. 3 is a diagram showing a method for delivering a drug such as a gene to a tissue or organ for transplant by use of plural laser beams;

FIG. 4 is a diagram showing a method for delivering a drug such as a gene to tissue or organ for transplant by irradiating the graft with a laser light from above and below;

FIG. 5 is a diagram showing a method for graft treatment;

FIG. 6 is a diagram showing a method for delivering a drug such as a gene to a tissue or organ for transplant in a state in which the graft is dipped in a solution containing the drug such as a gene;

FIG. 7 is a diagram showing a method for delivering a gene to skin for transplant;

FIG. 8 is a diagram showing the depth dependence of angiogenesis within the graft skin; and

FIG. 9 is a diagram showing an integrated value of neovascular regions within the graft skin.

FIG. 10 is a diagram of a laser-induced stress waves (LISW) gene delivering apparatus having an optical fiber.

FIG. 11 is a diagram showing the delivery efficiency of luciferase gene by using a laser-induced stress waves (LISW) gene delivering apparatus having an optical fiber.

### EXPLANATION OF THE CODES IN THE DRAWINGS

1 Graft of tissue or organ
2 Table
3 Light absorber
4 Transparent material
5 Lens
6 Laser light
7 Container
8 Solution containing a drug such as a gene
9 Terminal case with light absorber and transparent material
10 Optical fiber
11 Terminal case
12 Laser source
13 Shutter
14 Attenuator
15 Lens

### DETAILED DESCRIPTION OF THE PREFERRED EMBODIMENTS

The present invention relates to a method for delivering a drug such as a gene to a tissue or organ for transplant by use of a stress wave (pressure wave) induced by the laser light irradiation of a light absorber (laser-induced stress wave (LISW)) and thereby producing a high-performance tissue or organ for transplant.

Examples of the drug such as a gene delivered to a tissue or organ for transplant according to the present invention include nucleic acids such as gene DNA or RNA, proteins, sugars, polypeptides, and derivatives thereof. Other organic and inorganic compounds can also be used. In the present invention, the drug such as a gene used is a substance capable of exhibiting effects such as pharmaceutical effect within cells. Examples thereof include DNA or RNA available in gene therapy, biologically active substance-encoding DNA, an antisense strand of short-chain DNA or RNA or active derivatives thereof, ribozyme, and short-chain double stranded RNA (dsRNA). Examples of the short-chain double stranded RNA include a 15- to 30-base pair (bp), preferably 21- to 29-bp ribonucleic acid called small interfering RNA (siRNA). Alternatively, proteins such as biologically active substances may be used. Every pharmaceutical compound known in the art can be delivered as the drug of the present invention to cells. The present invention is particularly intended for a tissue or organ for transplant. Therefore, examples of the drug of the present invention include a drug such as a gene encoding a protein having an angiogenic effect, a drug such as a gene encoding a protein having an anti-infective effect, a drug such as a gene encoding a protein having a nerve regenerative effect, a drug such as a gene encoding a protein having a cell growth effect, and a drug such as a gene encoding a protein having a hematopoietic effect, from the viewpoint of enhancing the survival rate of a transplanted tissue or organ and preventing posttransplant infection. Examples of the drug such as a gene encoding a protein having an angiogenic effect include a hepatocyte growth factor (HGF), a platelet-derived wound healing factor (PDWHF), GM-CSF, EGF, TGF-a, HB-DGF, FGF, and VEGF or genes encoding them. Examples of the drug such as a gene encoding a protein having an anti-infective effect include IL-1, IL-2, IL-3, IL-4, IL-5, IL-6, IL-7, IL-8, IL-9, IL-10, IL-11, IL-12, IL-13, IL-14, IL-15, IL-16, IL-17, and IL-18 or genes encoding them. Examples of the drug such as a gene encoding a protein having a nerve regenerative effect include NGF, GDNF, and Midkine or genes encoding them. Examples of the drug such as a gene encoding a protein having a cell growth effect include EGF, TGF-a, HB-EGF, FGF, and HGF or genes encoding them. Examples of the drug such as a gene encoding a protein having a hematopoietic effect include GM-CSF, G-CSF, and erythropoietin or genes encoding them. Alternatively, genes encoding receptors of these drugs such as genes may be delivered. These drugs such as genes are taken as an illustration, and the present invention is not intended to be limited to them. The survival of a tissue or organ for transplant particularly requires the recirculation of the blood. In this regard, a drug such as a gene having an angiogenic effect is preferable.

The gene is incorporated in an expressible manner into an expression plasmid or vector comprising a promoter and so on and then delivered in an expressible manner into a tissue or organ for transplant.

In the present invention, the drug such as a gene can be delivered in an untreated state or after chemical modification to a tissue or organ for transplant. In the present invention, the chemical modification of the drug such as a gene refers to the complex formation between the drug to be delivered to a tissue or organ for transplant and a chemical substance capable of forming therewith a complex having a positive charge as a net charge. The complex having a positive charge accumulates around the cell of the tissue or organ for transplant. Therefore, when a laser-induced stress wave is applied thereto, the drug such as a gene to be delivered to the cell is efficiently delivered to the cell. In this context, the accumulation of the drug such as a gene around the cell refers to a state in which the drug such as a gene is easily delivered into the cell when the permeability of the cell membrane is increased, and also encompasses a state in which the drug is in contact with the cell membrane and a state in which the drug is present in no contact with the cell but in very close proximity to the cell. Examples of the chemical substance used in the chemical modification include lipids, polymers such as peptides, and outer envelope membranes having receptor-binding proteins. The lipids and the polymers such as peptides are substances having a positive charge. Any substance that has a positive charge and can form a complex with the drug to be delivered through the interaction therebetween may be used. Substances known as gene delivery reagents can be used. These substances, which are polymers having a positive charge, can also be called cationic polymers. Alternatively, a lipid bilayer vesicle (cationic liposome) comprising, as a component, a lipid having a positive charge can also be used. Specific examples thereof include polyethyleneimine (PEI, linear or branched) and commercially available gene delivery reagents. Examples of the commercially available gene delivery reagents include Lipofectamine (registered trademark), Lipofectamine plus (registered trademark), jet PEI (registered trademark), Oligofectamine (registered trademark), siLentFect (registered trademark), DMRIE-C (registered trademark), Transfectin-Lipid (registered trademark), and Effectene (registered trademark). Of them, polyethyleneimine (PEI) is classified into linear PEI and into branched PEI containing primary, secondary, or tertiary amine, both of which can be used. Moreover, PEI is not limited by molecular weight. Furthermore, PEI that has undergone chemical modification such as deacylation can also be used. Additional examples thereof can include basic substances such as arginine, polyarginine, poly-L-lysine, and chitosan. Further examples thereof can include cell-penetrating peptides such as Tat, and derivatives thereof, and nuclear transport signals such as NF-κβ. The use of an outer envelope membrane having a chain receptor-binding protein can deliver the drug to a cell having, on its surface, a receptor that can bind to the protein carried by the outer envelope membrane.

In the present invention, the chemically modified drug is also referred to as DNA-cationic liposome, DNA-polyethyleneimine (PEI), etc, by use of the name of the drug combined with the name of the chemical substance used in the chemical modification.

The above-described chemical substances having a positive charge may be used in combination of some of them.

A method for the chemical modification of the drug with a chemical substance is not particularly limited, and examples thereof include physical adsorption, chemical bond, and ionic bond. When a cationic liposome is used, the drug to be delivered to a tissue or organ for transplant may be incorporated within the liposome. When the drug chemically modified with the chemical substance(s) is applied to the cell, this chemically modified drug can accumulate around the cell of the tissue or organ for transplant through the electrostatic interaction therebetween.

In the present invention, the drug such as a gene is applied directly or after chemical modification to a tissue or organ for transplant, and a target comprising a light absorber or the light absorber combined with a transparent material, which is provided in proximity to the tissue or organ for transplant is irradiated with a pulse laser light. The pulse laser light is absorbed into the light absorber and induces a stress wave (pressure wave), resulting in an increase in the permeability of the membrane of the cell of the tissue or organ for transplant. As a result, the drug such as a gene is delivered into the cell. In this context, the application of the drug to a tissue or organ for transplant refers to the positioning of the drug in proximity to the cell of the tissue or organ for transplant through the contact between the drug and the tissue or organ for transplant so that the drug is delivered to the cell. Specifically, this application encompasses the injection, dropping, or application (coating) of the drug to the tissue or organ for transplant and the dipping of the tissue or organ for transplant into a solution containing the drug.

In the present invention, the tissue or organ for transplant to which the drug such as a gene is delivered is not limited and encompasses every tissue or organ capable of being transplanted. Examples thereof include skin, liver, kidney, lung, small intestine, pancreas, cornea, heart, colon, blood vessel. These tissues or organs may be collected from a living body or may be collected from a brain-dead donor or donor in cardiac arrest. Alternatively, an animal used is not limited and encompasses not only humans but also other mammals. When skin is used as a tissue for transplant, the skin for transplant may be a full thickness skin graft containing the whole skin layer or a split thickness skin graft containing a portion of the skin. The full thickness skin graft contains the dermis, while the split thickness skin graft does not contain much dermis. A graft obtained by increasing the area of a collected skin slice by culture can also be used. Preferably, skin collected from a patient himself or herself, which undergoes skin transplantation, is autografted as skin for transplant. Alternatively, allograft may be used. For example, a cultured skin slice from another person can be used. Furthermore, artificial skin for transplant produced on the basis of bioengineering may be used for the delivery. The artificial skin for transplant produced on the basis of bioengineering has a structure comprising animal skin-derived cells such as fibroblasts and keratinocytes within biocompatible polymer substances such as collagen, chitosan, chitin, fibrin, gelatin, hyaluronic acid, and chondroitin sulfate. According to the method of the present invention, the delivery of drugs such as a gene to animal cells within such alternative artificial skin can improve the survival rate of the alternative artificial skin. Examples of the alternative artificial skin include, but not limited to, those described in JP Patent Publication (Kokai) Nos. 2006-136326A (2006), 2004-121523A (2004), 2002-218971A (2002), 2001-104346A (2001), 2000-125855A (2000), 9-173362A (1997), and 5-317406A (1993), and JP Patent Publication (Kohyo) No. 11-503946A (1999).

The light absorber is used for absorbing a laser light and generating a stress wave. The light absorber is preferably a light absorber in a solid state, which has a high light-absorbing property and is made of, for example, a polymer or monomer such as rubber or resin, a metal, an amorphous substance such as an amorphous metal or alloy, or a crystal. A black light absorber may also be used. The light absorber preferably used is made of a material into which the laser light used is absorbed easily. Examples of the combination of the wavelength of the laser light used with an appropriate light absorber can include the combination of a 532- or 694-nm laser light with rubber (black, natural rubber), the combination of a 532-, 1064-, or 2100-nm laser light with a metal (aluminum, stainless, brass, nickel, etc.), and the combination of a 193-, 213-, 247-, 266-, 308-, or 355-nm laser light with resin.

Furthermore, a dye capable of absorbing a laser light can also be used as a light absorber. In this case, a tissue or organ to which the drug such as a gene is delivered is colored with a dye by applying the dye to the tissue or organ, and the colored portion may be irradiated with a laser light.

The light absorber may be used in combination with a transparent material that can confine therein plasma generated by the absorption of a laser into the light absorber and is capable of allowing the laser light with the wavelength used to pass therethough. The confinement of plasma in the transparent material can increase the impulse (time-integrated value of pressure) of a stress wave induced by a laser and more efficiently deliver the drug such as a gene to a tissue or organ for transplant. The transparent material may be put on the light absorber for use, for example, by affixing the transparent material to the light absorber. For example, a light absorber in a plate or sheet form and a transparent material in a plate or sheet form may be bonded together for use as a light-absorbing layer and a transparent layer, respectively. Any material for the transparent material that is transparent and can confine plasma can be used without limitations. The transparent material may be a solid material. Examples thereof include transparent resins such as polyethylene terephthalate (PET), polystyrene, PMMA, and polymethacryl/acrylamide, and glass such as quartz glass. The shape and size of the transparent material can be designed appropriately according to the light absorber. The thickness of the transparent material is, for example, 0.1 to several mm. The transparent material may be gel-like material such as polyvinyl alcohol gel and polyacrylamide gel, or liquid such as water and a buffer solution, or the like. When gel-like material or liquid is used, the impulse (time-integrated value of pressure) of laser-induced stress wave increases. Thus, the introduction of gene is accomplished with a laser light having lower energy than that used with solid transparent material.

Preferably, the graft of a tissue or organ for transplant is placed on a table with a high acoustic impedance made of a solid material and then irradiated with a laser. The table surface efficiently reflects the laser-induced stress wave, and the reflected wave acts on the graft. Therefore, the delivery effect of the gene or the like can be enhanced. Furthermore, the transparent material, which is made of a material with a high acoustic impedance, such as glass or an optical crystal, permits for the efficient multiple reflection of the laser-induced stress wave between the table surface and the undersurface of the transparent material. Therefore, the delivery effect of the gene or the like can be enhanced further. Examples of a material for the table include metals, ceramics, and reinforced plastics.

In the present invention, the light absorber or the light absorber combined with the transparent material by affixation or the like, which is intended for generating a laser-induced stress wave by laser light irradiation, is also referred to as a target.

The shape and size of the target is not limited and can be designed appropriately according to the state of a tissue or organ for transplant to which the drug such as a gene is delivered. Preferably, the area of the target is made equal to or larger than that of the graft of a tissue or organ for transplant. For example, for delivering the drug such as a gene to a skin graft, a target in a plate or sheet form can be used.

The light absorber is provided in proximity to a tissue or organ for transplant to which the drug such as a gene is delivered. Preferably, the light absorber is provided at a distance of 0 mm to 50 mm, preferably 0 mm to 10 mm, more preferably 0 mm to 5 mm, from the tissue or organ for transplant. This distance also corresponds to the distance between the tissue or organ for transplant to which the drug such as a gene is delivered and a laser light irradiation position. The thickness of the light absorber is, for example, 0.1 to several mm.

In the present invention, the selection of the pulse width, wavelength, intensity, and so on of a laser used for the irradiation of the light absorber is important for delivering the drug such as a gene to a tissue or organ for transplant. The application of excessive pressure damages the cell of the tissue or organ for transplant. These conditions must be set within a tolerance.

Any laser light that is capable of inducing a stress wave through absorption into the light absorber may be used without limitations. For example, a solid laser is indicated with an ion of an element generating a laser and the type of a base material holding the ion. Examples of the element include Ho (holmium), Tm (thulium), Er (erbium), and Nd (neodymium) belonging to rare earth. Examples of the base material include YAG, YSGG, and YVO. For example, Nd:YAG laser, Ho:YAG laser, Tm:YAG laser, Ho:YSGG laser, Tm:YSGG laser, Ho:YVO laser, and Tm:YVO laser can be used. Alternatively, XeCl laser can also be used as a gas laser. The laser light can be generated with a laser generator.

(1) Pulse width: The pulse width of the laser light used for the irradiation of the light absorber influences the efficiency of conversion from a laser energy to a stress. A pulse width longer than 500 ns has low efficiency of conversion from a laser energy to a stress. A pulse width shorter than 500 fs cannot expand plasma generated by the laser light irradiation of the light absorber and results in a stress wave merely generated for a short time. From these viewpoints, a pulse laser light having a pulse width of 500 fs to 500 ns is suitable as the laser light.
(2) Wavelength: The wavelength of the laser light used for the irradiation of the light absorber influences the efficiency of conversion from a laser energy to a stress. A laser light wavelength capable of being highly absorbed into the light absorber used is desirable. When a polymer, metal, or the like is used as a light absorber, a wavelength of 180 nm to 20 μm is suitable.
(3) Intensity: The intensity of the laser light used for the irradiation of the light absorber mainly influences the efficiency of delivery of the drug such as a gene and the safety of the cell of the tissue or organ for transplant. The selection of a laser wavelength is generally limited. Therefore, when the desired effect is expected, a laser fluence is mainly controlled. However, an excessive laser fluence causes the damage of the cell of the tissue or organ for transplant. Therefore, a Laser fluence of 0.1 mJ/cm² to 10 J/cm², preferably 0.1 J/cm² to 2 J/cm², is suitable for the method of the present invention. When an apparatus for delivering a drug to a tissue or organ having an optical fiber, the laser fluence may be low, for example, 0.1 J/cm² to 1 J/cm².

The size (area) of a spot irradiated with a laser light can be determined appropriately according to, for example, the size of the graft of a tissue or organ for transplant to which the drug such as a gene is delivered. The irradiated spot may be in a dotted or circular form. The size of the irradiated spot can be adjusted appropriately by changing the beam diameter of the laser light. In this case, the laser light irradiation may be carried out through a concentrating system such as a lens. The use of the concentrating system can appropriately adjust the position of laser irradiation and the size of the irradiated spot. In this case, the gene or the like can be delivered efficiently even to a graft with a large area by scanning the laser beam with a galvanometer mirror or the like. In this procedure, a cylindrical lens is used as a lens, with which the laser beam may be concentrated in a liner form. Alternatively, the scanning of a table for placing a graft thereon, instead of laser beam scanning, can be expected to produce similar effect. Furthermore, the use of plural laser beams can further reduce the time of delivery of the gene or the like. When plural laser beams are used, the simultaneous irradiation of the same site with the laser beams can generate the interference of the laser lights or laser-induced stress waves and enhance the efficiency of delivery of the gene or the like. Alternatively, the simultaneous laser irradiation of a graft from above and below can also produce the interference effect of the laser-induced stress waves and enhance the efficiency of delivery of the drug such as a gene or the like.

The stress wave generated from the light absorber is applied to the tissue or organ for transplant. In this context, the application of the stress wave to the tissue or organ for transplant refers to the propagation of the stress wave to the tissue or organ for transplant, which causes the contact between the stress wave and the cell in the tissue or organ for transplant, resulting in enhancement in the permeability of the cell membrane.

In the laser light irradiation, it is preferred for preventing the damage of the tissue or organ for transplant that only the stress wave should be brought into contact with the tissue or organ for transplant without directly irradiating the tissue or organ for transplant with the laser light.

The laser light may be irradiated to the tissue or organ through an optical fiber. In the method using the optical fiber, the light absorber can be fixed at the terminus of the optical fiber. The transparent material can be set on the light absorber. The light absorber and transparent material may be set in a case and the case can be fixed at the terminus of the optical fiber. The optical fiber may be a silica fiber or plastic fiber.

The method of the present invention has a high space control property during the delivery of the drug such as a gene and can also promote angiogenesis only in an arbitrary portion of the tissue or organ for transplant.

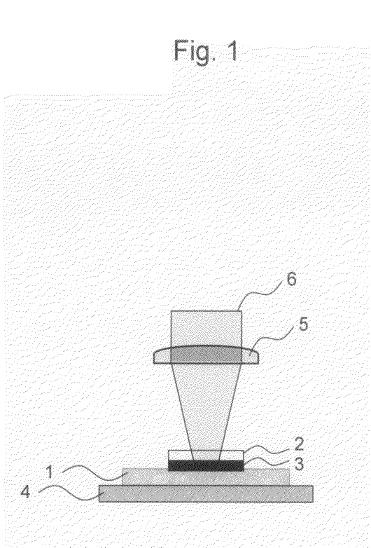

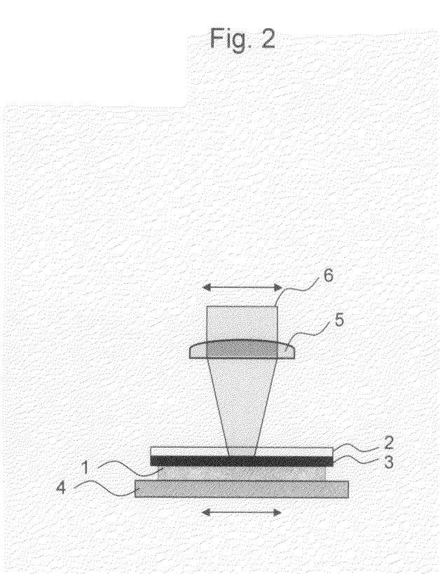

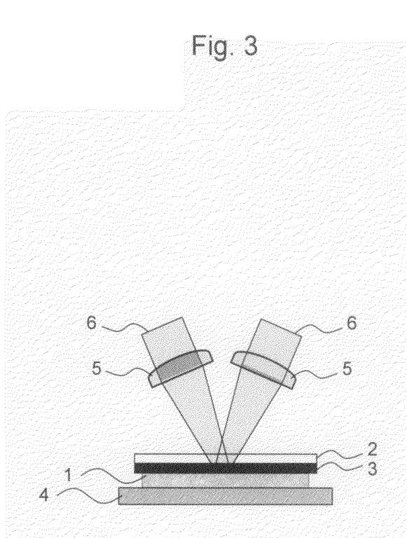

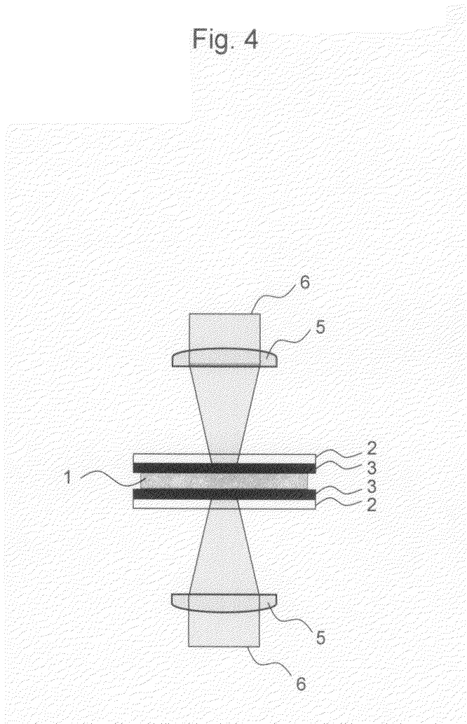

FIG. 1 shows the outline of the method for delivering a drug such as a gene to a tissue or organ for transplant according to the present invention. A graft 1 is placed on a table. A target comprising a transparent material 2 affixed to a light absorber 3 is placed on the graft 1. The target is irradiated with a laser light 6 through a concentrating system such as a lens 5. A laser-induced stress wave is generated by the target and falls on the graft 1. As a result, the drug such as a gene is delivered to the cell of the graft. FIG. 2 shows that, in the method shown in FIG. 1, the whole graft is irradiated with a laser light by laser beam scanning (indicated with the upper arrow) or by the scanning of the table (indicated with the lower arrow). As shown in FIG. 2, it is preferred for irradiating the whole graft with a laser light that the area of the target should be larger than that of the graft. FIG. 3 shows a method in which plural laser beams are used in the method for delivering a drug such as a gene to a tissue or organ for transplant. FIG. 4 shows a method in which the graft is irradiated with a laser light in both directions from above and below. The methods shown in FIGS. 3 and 4 cause the interference of the laser lights or laser-induced stress waves and allow for the easier delivery of the drug such as a gene to the tissue or organ for transplant.

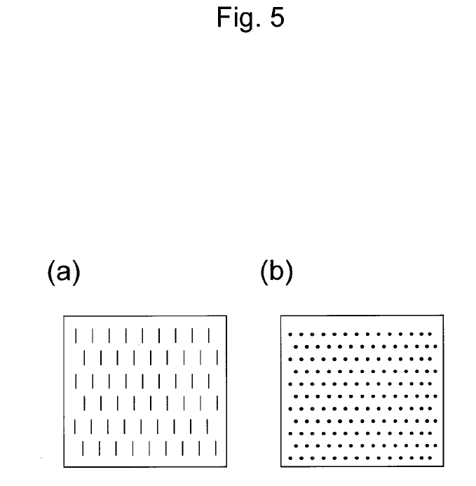

In the method of the present invention, a solution of a chemically modified or unmodified drug such as a gene is applied to the graft of a tissue or organ for transplant to which the drug such as a gene is delivered. The light absorber provided at a distance of 0 mm to 50 mm, preferably 0 mm to 10 mm, more preferably 0 mm to 5 mm, from the tissue or organ for transplant to which the drug such as a gene is delivered is irradiated with the laser light to thereby generate a stress wave. The generated stress wave falls on the cell of the graft, resulting in an increase in the permeability of the cell membrane. As a result, the chemically modified drug that has accumulated around the cell is delivered into the cell. The delivery of the drug such as a gene may be carried out by administering the drug into the graft by use of a syringe or the like or applying or dropping the drug to the graft. Alternatively, the graft may be dipped in advance in the drug. The graft, when having a large area, may be brought into contact with a buffer containing the drug such as a gene, after treatment for making incisions in the graft as shown in FIG. 5a or for making holes in the graft as shown in FIG. 5b. Such treatment can easily allow for the even, high-concentration penetration of the drug such as a gene within the graft having a large area and efficiently apply the drug such as a gene thereto. In this treatment, the size and density of the incisions or holes can be determined appropriately according to the size of the graft of the tissue or organ for transplant and the amount of the drug to be delivered.

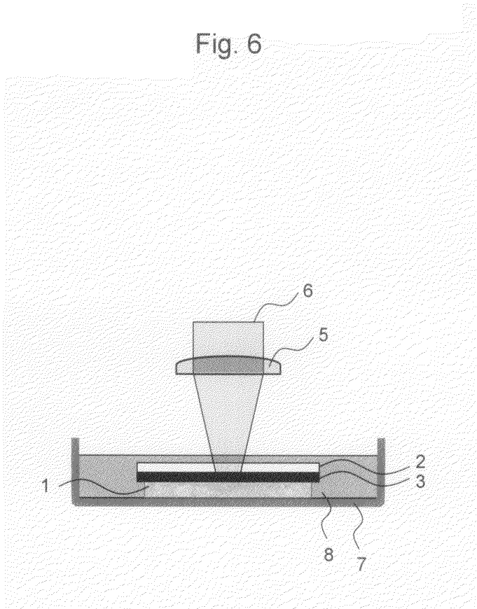

Furthermore, the laser irradiation may be performed, as shown in FIG. 6, in a state in which the graft is dipped in a buffer solution containing the drug such as a gene and is in contact therewith.

The present invention encompasses even an apparatus for delivering a drug such as a gene to a tissue or organ for transplant. The apparatus for delivery comprises: laser light irradiation means such as a pulse laser oscillator; an optical system such as a lens for controlling an irradiation area; a target comprising a light absorber or the light absorber combined with a transparent material; a drug such as a gene; and a table for placing a graft thereon. The pulse laser oscillator produces output through the oscillation of a pulse laser with the wavelength and pulse width described above. The apparatus is termed as a laser-induced stress waves (LISW) drug delivering apparatus or a laser-induced stress waves (LISW) gene delivering apparatus.

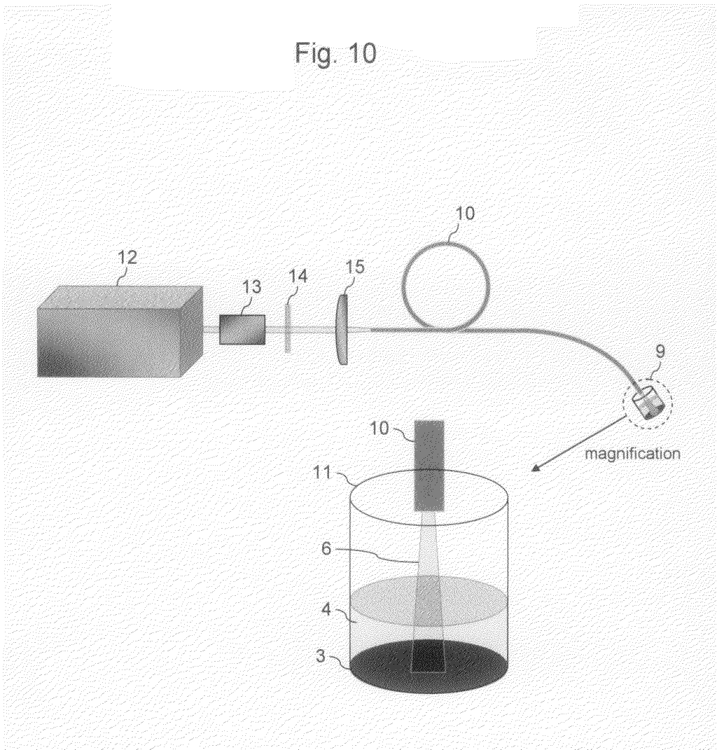

FIG. 10 shows the diagram of an apparatus for delivering a drug to a tissue of organ for transplant having an optical fiber 10. In the apparatus, a cylindrical terminal case 9 is fixed at the terminus of the optical fiber 10. The bottom panel of the case is made of a laser light absorbent (target) 3 and a transparent material 4 is set on the laser light absorber 3. Pulse laser light 6 irradiated from the optical fiber 10 is absorbed in the absorber 3 to induce plasma. The plasma is confined in the transparent material 4 and laser-induced stress waves (LISW) are efficiently induced. Although the transparent material 4 may be a solid material, gel-like material such as polyvinyl alcohol gel and polyacrylamide gel, or liquid such as water and a buffer solution is preferable. When gel-like material or liquid is used the impulse (time-integrated value of pressure) of laser-induced stress wave increases. Thus, the delivery of gene is accomplished with a laser light having lower energy than that used with solid transparent material. The higher energy of the laser light possibly destroy the optical fiber. Therefore, the laser light with the lower energy is advantageous for the apparatus using the optical fiber. The laser light is irradiated to the light absorber (target). The diameter of irradiated spot is not limited to, but preferably 2 mm to 20 mm, more preferably 2 mm which is the diameter of laser spot. The diameter of the terminal case comprising the light absorber and transparent material can be varied depend on the area to which genes are delivered.

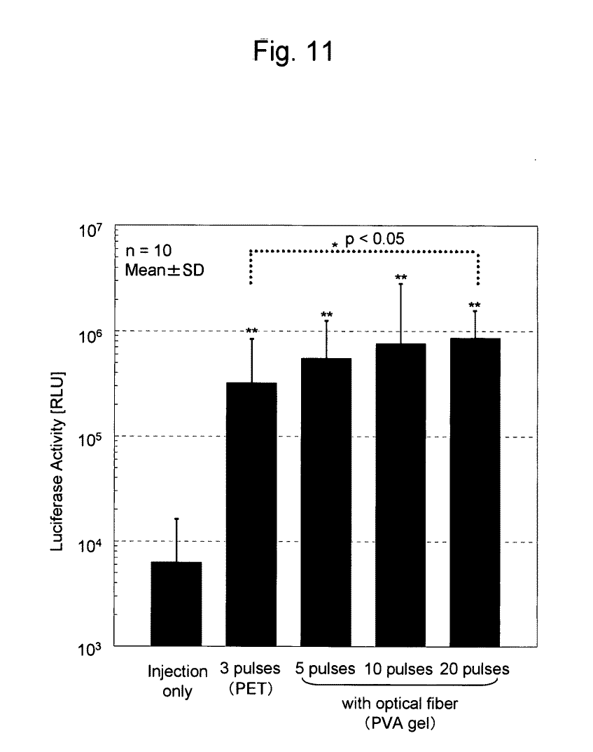

The apparatus with the optical fiber can be inserted into a lumen of an animal such as a digestive tract and blood vessel and deliver a drug to a tissue or organ in animal body in vivo. Especially, when the diameter of the case is about 2 mm, the apparatus can be used by inserting a thin vessel. Accordingly, the apparatus with an optical fiber enables endoscopic gene delivery by laser induced stress waves. The apparatus further comprises a laser light source 12, a shutter 13, an attenuator 14, a lens 15 and the like. When used as an endoscopic delivery apparatus, it may comprise means to inject a drug to the tissue or organ in an animal body. Alternatively, the drug may be administered to the tissue or organ previously. The present invention comprises an apparatus having an optical fiber as represented by FIG. 11 to deliver a drug such as a gene to a living tissue or organ in an animal body in vivo. The fiber is housed in a suitable protective tube, for example, a sheath or a catheter inserted in the sheath, and connected with the laser light source at the other end. In this catheter type apparatus, the terminal case comprising the light absorber and transparent material is placed at the terminus of the catheter. The present invention further comprises a method of delivering a drug such as a gene to a living tissue or organ in an animal body in vivo using the apparatus having an optical fiber as represented by FIG. 11. In this method, the tissue or organ includes liver, kidney, lung, small intestine, pancreas, cornea, heart, colon, blood vessel. Further, the tissue or organ which has disorder. For example, the tissue or included having cancer is included.

The apparatus of the present invention is not necessarily required to be provided at a site where a transplantation surgery is performed. For example, the apparatus is provided in an organ bank such as a tissue or skin bank, which provides a tissue or organ for transplant, and produces an enhanced, high-performance tissue or organ for transplant by imparting a variety of functions thereto. Then, the enhanced, high-performance tissue or organ for transplant can be transported to a medical institution. Moreover, the use of the apparatus of the present invention can automate procedures such as gene injection and laser irradiation involved in the production of a high-performance tissue or organ for transplant and allows for large-scale treatment and large-area treatment.

The present invention further encompasses a graft of a tissue or organ for transplant obtained by the method for delivering a drug such as a gene to a tissue or organ for transplant according to the present invention, wherein the graft of a tissue or organ for transplant has particular function such as angiogenic effect imparted or enhanced by the delivery of a drug such as a gene that imparts the function thereto. Examples thereof include a high-performance tissue or organ, specifically, high-performance skin for transplant, enhanced in angiogenic ability by HGF delivery by the method of the present invention. The enhanced angiogenic function promotes graft survival. Specifically, the tissue or organ for transplant produced by the method of the present invention has a survival ability larger than that of a tissue or organ for transplant to which a drug such as the gene that imparts particular function such as angiogenic effect is not delivered. The survival ability can be determined, for example, by transplanting the graft of a tissue or organ for transplant to be transplanted to plural individuals of experimental animals such as immunodeficient mice and measuring a survival rate after a lapse of a given time. The method for drug delivery of the present invention can deliver a gene without use of virus vectors. Therefore, the produced tissue or organ for transplant contains no virus-derived substance and is safe to human bodies. Furthermore, the method for drug delivery of the present invention can deliver a drug even to a large-size graft evenly and efficiently. Therefore, the produced tissue or organ for transplant is a graft that comprises the drug distributed evenly and has particular function imparted to the whole graft. Furthermore, the graft of the tissue or organ for transplant produced by the method of the present invention sometimes has incisions or holes for delivering the drug such as a gene.

The high-performance tissue or organ obtained by the method of the present invention can be transplanted into a patient in need of transplantation by a tissue or organ transplantation method known in the art. When the tissue or organ for transplant is skin, the skin may be transplanted in combination with artificial skin such as artificial dermis containing a biocompatible material such as chitin, chitosan, or collagen.

### EXAMPLE

The present invention will be described specifically with reference to Example below. However, the present invention is not intended to be limited to this Example.

### Example 1

It was found that the transport/delivery of Hepatocyte Growth Factor (HGF)-expressing gene into skin for transplant promotes angiogenesis within the transplanted skin.

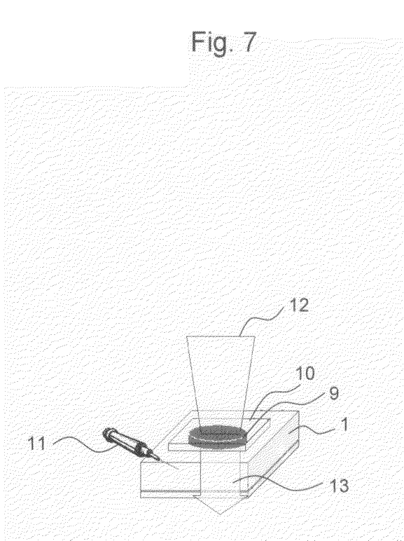

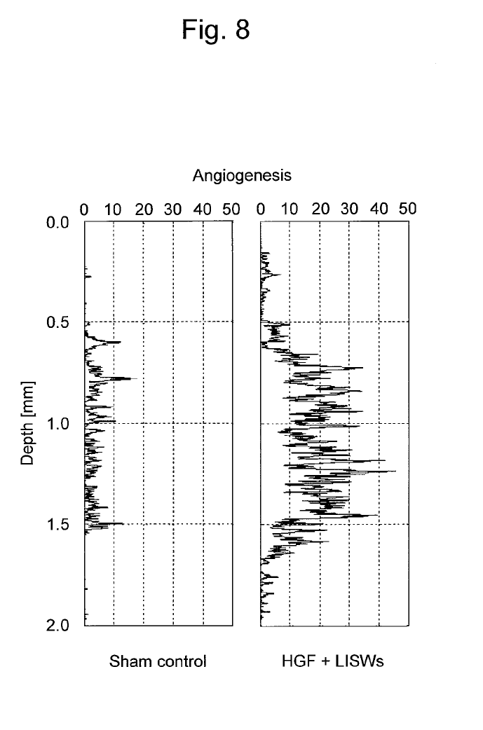

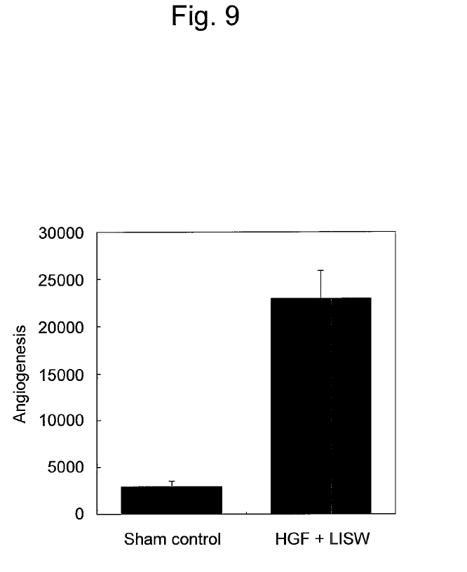

The gene for delivery used was an Invitrogen pcDNA3.1 plasmid vector in which human HGF cDNA was encoded at the NotI site. A 2 cm×2 cm skin slice including subcutaneous tissues was collected from the back of an eleven-week-old Sprague-Dawley rat (body weight 300 to 380 g) and used as a graft after the removal of subcutaneous fat. The HGF-expressing gene plasmid vector (concentration 1.0 μg/μl) was injected in an amount of 10 μl into the skin from behind of the graft by use of a syringe. A light absorber was provided on the dermis layer side. The construction of gene transfer is shown in FIG. 7. The light absorber used was polyethylene terephthalate of 1.0 mm in thickness bonded to black, natural rubber of 0.5 mm in thickness. Silicon vacuum grease was applied to between the target and the graft to thereby carry out acoustic impedance matching. After the loading of the target, the second harmonics (wavelength 532 nm, pulse width 6.0 ns (FWHM)) of Q-switched Nd:YAG laser was concentrated at a diameter of 3 mm onto the target by means of a flat convex lens (f=170 mm). Since the whole energy of the laser is absorbed into the target, the laser does not directly interact with the skin tissue. The laser fluence was set to 1.2 J/cm². Laser irradiation was carried out with 3 pulses by replacing the target for each of the pulses. After irradiation, the graft was affixed and sutured to the site from which it was collected. Three days after transplantation, the transplanted skin was subjected to biopsy. Neovascular regions within the transplanted skin were identified by means of immunostaining with an anti-CD31 antibody. The stained sample was observed under bright field microscopy. The range of RGB indicated by the pixel of the stained region was determined from the obtained image. An average value from 5 rats was determined under each condition (n=5). The depth dependence of angiogenesis within the transplanted skin is shown in FIG. 8. The longitudinal axis denotes a depth from the skin surface, and the horizontal axis denotes neovascular regions within the transplanted skin. Moreover, FIG. 9 is a graph showing the integration of the neovascular regions shown in FIG. 8. A condition under which a skin slice excised from the rat skin was transplanted again to the rat was used as a sham control. A condition under which a skin slice excised from the rat skin, to which the HGF-expressing gene plasmid vector was injected, was transplanted after the application of a stress wave thereto was designated as HGF+LISW. The HGF+LISW resulted in a significantly larger number of new blood vessels within the transplanted skin than the sham control. High-density angiogenesis was observed from 0.7 mm to 1.5 mm in skin depth. This result demonstrates that the present invention can produce high-performance skin for transplant (super skin).

### Example 2

The gene delivery into rat skin was performed using the apparatus of FIG. 10. Second harmonic of Nd:YAG laser (Wavelength: 532 nm, Pulse width: 6 ns) was used as a laser light source, step index type silica optical fiber having core diameter of 1 mm and length of 1 m was used as an optical fiber, black natural rubber was used as a light absorber (target) and polyvinyl alcohol (PVA) gel was used as a transparent material. Plasmid DNA comprising luciferase gene was used as a gene. The plasmid was injected in rat skin and the bottom of the cylindrical case of the apparatus was contacted with the skin. The laser light was irradiated to the absorber (target). The diameter of the irradiated spot on the target was 2 mm, laser fluence was 0.7 J/cm² and the number of irradiated pulses was 5, 10 or 20. After the laser was irradiated, luciferase activity in the skin was measured. The rat skin was removed and homogenized. The luminescence of the homogenate was measured using a luminometer to determine the luciferase activity.

FIG. 11 shows the luciferase activity of the skin. FIG. 11 also shows the comparative experiments in which plasmid DNA was injected in the skin but no laser light was irradiated, or laser light was irradiated without using the optical fiber (Transparent material was PET and laser fluence was 1.2 J/cm²). As shown in FIG. 11, the gene delivery efficiency using the apparatus with the optical fiber is the same with that using an apparatus without an optical fiber.

The apparatus of FIG. 10 enables laser light be irradiated with flexibility and therefore gene delivery can be performed with high efficiency.

What is claimed is:

1. A method of producing a graft of a tissue or organ for transplant having promoted survival ability which comprises delivering a drug to an animal tissue or organ in vitro by applying the drug to the tissue or organ, irradiating a pulse laser light to a light absorber which is provided in proximity to the tissue or organ to induce a stress wave and applying the stress wave to the tissue or organ, wherein the drug is selected from the group consisting of a gene encoding a protein having an angiogenic effect, a gene encoding a protein having an anti-infective effect, a gene encoding a protein having a nerve regenerative effect, a gene encoding a protein having a cell growth effect, and a gene encoding a protein having a hematopoietic effect.

2. The method of producing a graft of a tissue or organ for transplant having promoted survival ability according to claim 1, wherein the drug is chemically modified with cationic polymer or cationic liposome.

3. The method of producing a graft of a tissue or organ for transplant having promoted survival ability according to claim 1, wherein the animal tissue or organ is selected from the group consisting of skin, liver, kidney, lung, small intestine, pancreas, and cornea collected from the animal body.

4. The method of producing a graft of a tissue or organ for transplant having promoted survival ability according to claim 1, wherein the light absorber is selected from the group consisting of rubber, resin or metal.

5. The method of producing a graft of a tissue or organ for transplant having promoted survival ability according to claim 1, wherein the light absorber is used in combination with a transparent material.

6. The method of producing a graft of a tissue or organ for transplant having promoted survival ability according to claim 5, wherein the transparent material is selected from the group consisting of a transparent resin, glass, gel and liquid.

7. The method of producing a graft of a tissue or organ for transplant having promoted survival ability according to claim 1, wherein the graft is treated for making incisions or holes such that the drug can be easily penetrated into the graft.

8. A graft of a tissue or organ for transplant produced by delivering a drug to the tissue or organ by the method of producing a graft of a tissue or organ for transplant having enhanced survival ability according to any one of claims 1 to 7, wherein the drug is selected from the group consisting of a gene encoding a protein having an angiogenic effect, a gene encoding a protein having an anti-infective effect, a gene encoding a protein having a nerve regenerative effect, a gene encoding a protein having a cell growth effect, and a gene encoding a protein having a hematopoietic effect and the gene is incorporated in an expressible manner to the tissue or organ.

9. The graft of a tissue or organ for transplant according to claim 8, wherein the angiogenic ability is promoted.

10. The graft of a tissue or organ for transplant according to claim 8, wherein the survival ability is promoted.

11. The graft of a tissue or organ for transplant according to claim 8, wherein the tissue or organ is selected from the group consisting of skin, liver, kidney, lung, small intestine, pancreas, and cornea collected from the animal body.

12. An apparatus for delivering a drug to a graft of a tissue or organ comprising
a drug to be delivered to the graft of the tissue or organ,
a target comprising a light absorber or the light absorber combined with a transparent material,
a means for irradiating a laser light,
wherein laser light is irradiated to the target to generate a stress wave from the light absorber, and applying the stress wave to the graft of the tissue or organ to deliver the drug to the graft of the tissue or organ and the drug is selected from the group consisting of a gene encoding a protein having an angiogenic effect, a gene encoding a protein having an anti-infective effect, a gene encoding a protein having a nerve regenerative effect, a gene encoding a protein having a cell growth effect, and a gene encoding a protein having a hematopoietic effect.

13. The apparatus for delivering a drug to a graft of a tissue or organ according to claim 12, further comprising an optical fiber having a terminal case at the terminus of the optical fiber, wherein the terminal case has the target on the bottom of the case in which the transparent material is set on the light absorber.

14. The apparatus for delivering a drug to a graft of a tissue or organ according to claim 12 or 13, wherein the drug is chemically modified with cationic polymer or cationic liposome.

15. The apparatus for delivering a drug to a graft of a tissue or organ according to claim 12 or 13, wherein the light absorber is selected from the group consisting of rubber, resin or metal.

16. The apparatus for delivering a drug to a graft of a tissue or organ according to claim 12 or 13, wherein the light absorber is used in combination with a transparent material.

17. The apparatus for delivering a drug to a graft of a tissue or organ according to claim 12 or 13, wherein the transparent material is selected from the group consisting of a transparent resin, glass, gel and liquid.

18. The apparatus for delivering a drug to a graft of a tissue or organ according to claim 12 or 13, wherein the animal tissue or organ is selected from the group consisting of skin, liver, kidney, lung, small intestine, pancreas, and cornea collected from the animal body.

19. An apparatus for delivering a drug to a tissue or organ in vivo comprising
a target comprising a light absorber or the light absorber combined with a transparent material,
a means for irradiating a laser light,
an optical fiber having a terminal case at the terminus of the optical fiber,
wherein the terminal case has the target on the bottom of the case and the transparent material is set on the light absorber and the drug is selected from the group consisting of a gene encoding a protein having an angiogenic effect, a gene encoding a protein having an anti-infective effect, a gene encoding a protein having a nerve regenerative effect, a gene encoding a protein having a cell growth effect, and a gene encoding a protein having a hematopoietic effect.

20. A method for delivering a drug to a tissue or organ in vivo using the apparatus of claim 19, which comprises applying a drug to the tissue or organ and irradiating laser light to the tissue or organ in vivo, wherein the drug is selected from the group consisting of a gene encoding a protein having an angiogenic effect, a gene encoding a protein having an anti-infective effect, a gene encoding a protein having a nerve regenerative effect, a gene encoding a protein having a cell growth effect, and a gene encoding a protein having a hematopoietic effect.
